(19) United States
(12) Patent Application Publication
(10) Pub. No.: US 2007/0243261 A1
(43) Pub. Date: Oct. 18, 2007

(54) Biocompatible microcapsules

(76) Inventor: Peter R. Bahn, Mt. Vernon, IL (US)

Correspondence Address: Peter R. Bahn, 10415 E. Boyd Rd., Mt. Vernon, IL, 62864, US

(21) Appl. No.: 11/724,579

(22) Filed: Mar. 16, 2007

Related U.S. Application Data
(60) Provisional application No. 60/791,966, filed on Apr. 14, 2006.

(51) Int. Cl. A61K 9/50; B01J 13/02

(52) U.S. Cl. 424/491; 264/4.1

(57) ABSTRACT

One of the most prominent amino acids found in biological proteins is aspartic acid. In the present invention L-aspartic acid was heated at 150 deg C for up to 100 hrs to form thermal polyaspartylimide which when heated in boiling water without addition of base hydrolyzed to form thermal polyaspartic acid which upon cooling formed protective biocompatible microcapsules.

### REFERENCES

Bahn, P. R. and Pappelis, A.: 2005a, Thermal Polyaspartic Acid Microspheres, ISSOL 2005 Abstracts, P-50, 70.
Bahn, P. R., Pappelis, A. and Bozzola, J.: 2005b, Protocell-Like Microspheres From Thermal Polyaspartic Acid, National Workshop on Astrobiology, Capri, Italy.
Bahn, P. R., Pappelis, A. and Bozzola, J.: 2006, Origins of Life and Evolution of the Biopshere, 36, 617-619
Fox, S. W. and Bahn, P. R.: 1994, Thermal Polyamino Acids in the Origins of Life and Biomedical Applications, in Bittar, E. E. and Bittar, N. (eds), Principles of Medical Biology, Vol. 1B, Evolutionary Biology, JAI Press Inc., London, 73-85
Fox, S. W. and Harada, K.: 1958, Thermal Copolymerization of Amino Acids to a Product Resembling Protein, Science 128, 1214.
Fox, S. W. and Harada, K.: 1960, The Thermal Copolymerization of Amino Acids Common to Protein, Journal of the American Chemical Society 82, 3745-3751.
Fox, S. W. and Harada, K.: 1962, Thermal Polymerization of Amino Acid Mixtures Containing Aspartic Acid or a Thermal Precursor of Aspartic Acid, U.S. Pat. No. 3,052,655.
Fox, S. W. and Harada, K.: 1963, Method of Making Copolymers of Amino Acids Containing Glutamic Acid, U.S. Pat. No. 3,076,790.
Fox, S. W., Harada, K. and Kendrick, J.: 1959, Production of spherules from Synthetic Proteinoid and Hot Water, Science 129, 1221-1223.
Fox, S. W. and Veltri, R. W.: 1990, Microencapsulated Antitumor Agent, U.S. Pat. No. 4,963,364.
Schiff, H.: 1897, Ueber Polyaspartsaeuren, Berichte der Deutschen Chemiscthen Gesellschraft XXX 16 (S 107) A.
Schiff, H.: 1898, Ueber Polyaspartsaeuren, Annalen der Chemie 303 (S 112) A.
Schiff, H.: 1899a, Ueber Polyaspartsaeuren, Annalen der Chemie 307 (S 115) A.
Schiff, H.: 1899b, Ueber Polyaspartsaeuren, Annalen der Chemie 310 (S 116) A.
Steiner, S. S.: 1990, Anhydrous Delivery Systems for Pharmacological Agents, U.S. Pat. No. 4,976,968.
Steiner, S. S. and Rosen, R.: 1990, Delivery Systems for Pharmacological Agents Encapsulated With Proteinoids, U.S. Pat. No. 4,925,673.
Vegotsky, A., Harada, K. and Fox, S. W.: 1958, The Characterization of Polyaspartic Acid and Some Related Compounds, Journal of the American Chemical Society 80, 3361-3366.

### INTRODUCTORY DATA

This regular utility patent application is based on the provisional patent application entitled Biocompatible Microcapsules, Application No. 60/791,966 with a Filing Date of Apr. 13, 2006 filed by the Applicant Peter R. Bahn and with the Confirmation Number 9426.

### BACKGROUND OF THE INVENTION

Thermal polyaspartic acid was the first thermal polypeptide to be synthesized in a chemist's laboratory, by Hugo Schiff (1897, 1898, 1899a, b). Other thermal polyamino acids were first synthesized in a laboratory by Sidney W. Fox and colleagues by heating mixtures of amino acids with large amounts of aspartic acid and glutamic acid (Fox and Harada, 1960, 1962, 1963). The term “proteinoids”, for such thermal polyamino acids, was coined by Fox in 1956. Thermal polyamino acids, which are branched polymers, were referred to as thermal proteins by Chemical Abstracts in 1972. This discovery of how to make thermal proteins was followed by the discovery that these thermal branched proteins, when heated in boiling water and allowed to cool to room temperature, formed microcapsules (Fox, Harada, and Kendrick, 1959). Apparently, modern linear biological proteins do not exhibit this attribute of forming microcapsules.

Thermal polyamino acids have been fed to rodents and bacteria which thrived on such nutrients (Fox and Harada, 1960). Hence, thermal proteins which contain a predominance of aspartic and glutamic acids are biologically compatible and have found a number of biomedical applications (Fox and Bahn, 1994) including the protective microencapsulation of pharmaceuticals as delivery vehicles for such pharmaceuticals (Fox and Veltri, 1990; Steiner and Rosen, 1990; Steiner, 1990).

Fox and his colleagues always prepared microcapsules from heteropolyamino acids and believed that such heterogeneity was necessary for the formation of microcapsule structure (Fox, Harada, and Kendrick, 1959). However, this assumption has turned out not to be the case. It was recently reported at a science conference, based on unreported research previously done by the present inventor, that commercially prepared thermal polyaspartic acid, which is a monopolymer, does indeed form microspheres when heated in boiling water and allowed to cool to room temperature (Bahn and Pappelis, 2005). In a new experiment, it was of interest to prepare thermal polyaspartic acid in the laboratory from scratch and to prepare microcapsules therefrom.

Thermal polyaspartic acid is a polymer which has been known for at least 100 years and is also known to be thoroughly compatible with all known biological systems. Among the many current commercial uses of polyaspartic acid are its use as absorbants and fillers in baby diapers, womens' cosmetics, and toothpaste. However, it was not previously known prior to the discovery reported here that thermal polyaspartic acid could be used to make microcapsules as protective, biologically compatible delivery systems for pharmaceuticals and other possible commercial uses such as biodegradable protective agents for any type of sensitive material encapsulated within such protective, biologically compatible microcapsules.

### MATERIALS AND METHODS

Thermal polyaspartylimides were synthesized in our laboratory as follows: Into each of four capped topped glass vials was placed 10 g of L-aspartic acid from Research Organics, Inc. The vials of aspartic acid were heated in a drying oven at 180 deg C. for 0, 10, 50, and 100 hrs respectively in the dark without being disturbed. At the end of their respective heating times, each vial was allowed was allowed to cool to room temperature, and then capped and rotated three times before storage.

Thermal polyaspartylimides were converted to thermal polyaspartic acids, and thence assembled into microcapsules as follows: From each of the four vials, 10 mg of their contents were placed in each of four glass test tubes. The test tubes were shaken as they were heated in a low flame alcohol lamp to cause the mixtures to flash boil. The test tubes were allowed to cool to room temperature. Then two to three drops from each of the test tubes were placed on microscope slides. The microscope slides were either viewed with an optical light microscope or the microscope slides were air dried at 45 deg C. for 15 min and were then viewed with a scanning electron microscope.

### RESULTS

Aspartic acid is a white crystalline powder. Upon heating at 150 deg C., this white powder slowly turns into a slightly pinkish tan color so that the white powder turns very slightly pinkish at 10 hrs, somewhat more at 50 hrs, and yet still a bit more at 100 hrs. During this time the aspartic acid does not undergo a phase change. The white powder does not melt, but rather, the white powder simply changes color to a very light pinkish tan color. This heating of aspartic acid catalyzes the dehydration condensation polymerization of the aspartic acid into the polymer thermal polyaspartylimide. Thermal polyaspartylimide has a different electronic absorption spectrum than aspartic acid, which accounts for the slightly pinkish tan color of thermal polyaspartylimide.

When thermal polyaspartylimide was placed in water and the water boiled, the slightly pinkish tan material went into solution and lost its pinkish tan tinge. This procedure accomplished the aqueous hydrolysis of the thermal polyaspartylimide (i.e. the opening of its internal imide rings) into thermal polyaspartic acid. During hydrolysis, the internal imide rings of thermal polyaspartylimide can open up in two different ways so as to form alpha linkages or beta linkages between successive aspartic acid residues in the polymer.

When the thermal polyaspartic acid in the boiling water was allowed to cool to room temperature, biocompatible microcapsules comprised of thermal polyaspartic acid coalesced out of solution. With polymers described here, the numbers of microcapsules produced were least in the 10 hr heating treatment and greatest in the 50 hr and 100 hr treatments. In this present experiment, microcapsules were not observed in the unheated (0 hr) aspartic acid flash boiled control. The microcapsules have diameters that are generally about 1.0 micron, with a range of approximately 0.2-1.5 micron. The thermal polyaspartic acid microspheres are probably held together by hydrogen-bonded twinned dicarboxylic acid pairs between neighboring peptide chains. Evidence for this theory is provided by the fact that when thermal polyaspartic acid microspheres, which in suspension, have a pH of about 3.0 are subjected to basic conditions by the drop-wise addition of potassium hydroxide solution, the thermal polyaspartic acid microspheres dissolve by the time that a pH of about 11.0 is reached. That is, protonated carboxylic acid groups would attract each other by hydrogen bonding between the said carboxylic acid groups but unprotonated, negatively charged carboxylic acid groups would repel each other because of their mutual negative charges.

### MICROENCAPSULATION

It is a well-known fact from all of the prior art that thermal proteins, when heated in hot water and allowed to cool to room temperature microencapsulate whatever chemical compounds are present in solution as the thermal proteins are forming microcapsules. Chemical compounds outside the thermal protein microcapsules can be removed by washing the thermal protein microcapsules several times in a centrifuge. The microcapsules can be stored in solution or can be air dried and stored indefinitely without deterioration.

As an example of how to microencapsulate various molecules, this inventor did the following experiment to microencapsulate RNA inside the thermal polyaspartic acid microcapsules: 50 mg of polyaspartic acid (from L-aspartic acid which had bee heated to 180 deg for 50 hrs as indicated above) and 50 mg of the sodium salt of ribonucleic acid from yeast (purchased from ICN Biomedicals, Inc.) were placed in a test tube with 5 ml of distilled water. The mixture was heated to boiling over a flame whereupon much of the thermal polyaspartic acid and all of the RNA went into solution. The solution was allowed to cool to room temperature slowly. As the solution cooled down, it became turbid. When this turbid solution was examined under a microscope at 1000×, thermal polyaspartic acid microspheres containing RNA were seen. The turbid suspension of RNA containing microspheres was decanted into another test tube. Overnight, the RNA containing microspheres settled on the bottom and sides of the test tube leaving a clear solution of RNA containing liquid above the microspheres. This exterior RNA was washed away by allowing the RNA microspheres to settle for five successive nights, discarding the liquid above, and resuspending the microspheres to a volume of 5 mls with fresh distilled water. This is the same method which can be used to microencapsulate any chemicals inside thermal polyaspartic acid or thermal protein microspheres is such thermal protein (i.e. proteinoid) is capable of forming microspheres.

What is claimed are:

1. Protective microcapsules comprised of thermal polymers of a single biologically compatible monomer.

2. Protective microcapsules as described in claim 1 wherein the said monomer is an amino acid.

3. Protective microcapsules as described in claim 2 wherein the said amino acid is a dicarboxylic amino acid.

4. Protective microcapsules as described in claim 3 wherein the said dicarboxylic amino is an alpha amino acid.

5. Protective microcapsules as described in claim 4 wherein the said alpha amino acid is one of the twenty naturally occurring amino acids the residues of which are found in biological proteins.

6. Protective microcapsules as described in claim 5 wherein the said naturally occurring amino acid is aspartic acid.

7. Protective microcapsules as described in claim 6 wherein the said aspartic is L-aspartic acid.

8. A method for making protective microcapsules comprising thermally polymerizing a single biologically compatible monomer, boiling the resulting thermal polymers in water, and allowing the thermal polymers to cool.

9. A method for making protective microcapsules as described in claim 8 wherein the said monomer is an amino acid.

10. A method for making protective microcapsules as described in claim 9 wherein the said amino acid is a dicarboxylic amino acid.

11. A method for making protective microcapsules as described in claim 10 wherein the said dicarboxylic amino acid is an alpha amino acid.

12. A method for making protective microcapsules as described in claim 11 wherein the said alpha amino acid is one of the twenty naturally occurring amino acids the residues of which are found in biological proteins.

13. A method for making protective microcapsules as described in claim 12 wherein the said naturally occurring amino acid is aspartic acid.

14. A method for making protective microcapsules as described in claim 13 wherein the said aspartic acid is L-aspartic acid.

15. A method of microencapsulating a chemical substance by heating the said chemical substance with thermal polyaspartic acid in water and allowing the water to corn to room temperature, and then washing the exterior resulting said chemical substance away from the resulting thermal polyaspartic acid microspheres containing the said chemical substance.

16. A method of microencapsulating a chemical substance as described in claim 15 wherein that said chemical substance is a pharmacologically useful substance.

17. A method of microencapsulating a chemical substance as described in claim 16 wherein the said chemical substance is nucleic acid.

18. A method of microencapsulating a chemical substance as described in claim 17 wherein the said nucleic acid is RNA.
